(19) United States
(12) Patent Application Publication
(10) Pub. No.: US 2023/0140385 A1
(43) Pub. Date: May 4, 2023

(54) BLOOD PRODUCT STORAGE SYSTEM WITH SEALABLE BAG

(71) Applicant: Rich Technologies Holding Company, LLC, Buffalo, NY (US)

(72) Inventor: Ilya Ilyin, Wayland, MS (US)

(21) Appl. No.: 17/978,692

(22) Filed: Nov. 1, 2022

Related U.S. Application Data
(60) Provisional application No. 63/274,356, filed on Nov. 1, 2021.

(51) Int. Cl. A61J 1/14; A61J 1/10; A61J 1/20

(52) U.S. Cl. CPC A61J 1/1468; A61J 1/10; A61J 1/1475; A61J 1/2037

(57) ABSTRACT

A system for storing a blood product includes an inner container configured to contain the blood product, wherein the inner container is permeable to a gas system, and an outer container including a first end, a second end opposite the first end, a cavity defined between the first and second ends, an inlet in fluid communication with the cavity, and a valve operable to control a flow of the gas system through the inlet. The inner container is insertable into the cavity through the first end, the first end is sealable to hermetically seal the inner container within the cavity, and the outer container is made of a material that is impermeable to the gas system.

### BACKGROUND

The present invention relates to blood product storage systems, and more particularly to bags and containers for storing a blood product in a controlled atmosphere.

A method and device for preserving blood or its components in a gas medium under pressure and system for the same is described in PCT Publication No. WO2012/177820, which is incorporated herein by reference in its entirety. According to the '820 publication, the blood or blood components are placed in a bag that is made of a xenon gas-permeable material. The bag is then placed into a hermetically-sealed cylindrical chamber into which xenon-containing gas (with a xenon content of at least 65 vol. %) is fed under pressure until the pressure in the chamber reaches the approximate of 3.5 to 5 bars, after which the chamber is placed in storage at a temperature within the range from 3-6° C. Bags that are made of the gas-permeable material are designed to allow xenon to pass through the bag for the implementation of this method. In this method, the xenon-containing gas (fed under pressure into the chamber) passes through the bag wall, after which the blood or blood components in the bag are partially or fully saturated with xenon.

Movement of the chamber that contains the pressurized gas can be cumbersome between users, for example, between a blood bank facility and a hospital. In addition, such a chamber may require a significant amount of gas to be pumped into the container in order to create suitably high pressures. According to another method, described in U.S. Publication No. 2018/0249703, which is incorporated herein by reference in its entirety, the gas-permeable bag containing the blood or blood components is placed into a secondary bag, which in turn is filled with a xenon-containing gas system. This “bag-in-bag” assembly is then placed into a pressure chamber, and a second gas, which may be ordinary compressed air, is introduced into the chamber to elevate the pressure of the xenon-containing gas system in the secondary bag and in turn cause the xenon to pass through the gas-permeable bag.

It has been found, however, that filling the secondary bag with the xenon-containing gas system and preventing subsequent leaking of the gas system can be difficult to accomplish. For example, existing one-way gas fill valves tend to be relatively expensive for use in a disposable product, complex to manufacture, and are difficult to properly seal with the thin walls of the secondary bag. In view of the current state of the art, there remains a need for a device which provides storage conditions for preserving blood products and cellular cultures in a gas medium under pressure that is reliable, inexpensive, and easy to use in the blood bank and hospital environment.

### SUMMARY

The present disclosure provides, in one aspect, a system for storing a blood product including an inner container configured to contain the blood product, wherein the inner container is permeable to a gas system, and an outer container including a first end, a second end opposite the first end, a cavity defined between the first and second ends, an inlet in fluid communication with the cavity, and a valve operable to control a flow of the gas system through the inlet. The inner container is insertable into the cavity through the first end, the first end is sealable to hermetically seal the inner container within the cavity, and the outer container is made of a material that is impermeable to the gas system.

In some embodiments, the valve includes a sleeve extending into the cavity and having a perforation, and the gas system is configured to flow into the cavity through the sleeve and the perforation when a gas system source is connected to the inlet.

In some embodiments, the sleeve is configured to collapse when a pressure within the cavity is greater than a pressure at the inlet.

In some embodiments, the outer container includes a first sheet of material and a second sheet of material sealed together at the second end, and the inlet extends through the second end.

In some embodiments, the valve includes a flexible membrane and a orifice formed in the flexible membrane.

In some embodiments, the membrane is configured to expand to open the orifice when a gas system source is connected to the inlet.

In some embodiments, outer container includes a transparent window.

In some embodiments, the inner bag is visible through the transparent window when the inner bag is hermetically sealed within the cavity.

In some embodiments, the valve includes external threads.

In some embodiments, the first end of the outer container includes an interlocking closure.

In some embodiments, the gas system includes xenon.

In some embodiments, the outer container comprises metal foil.

In some embodiments, the valve is sealed inside the cavity when the first end is sealed.

In some embodiments, a compartment is disposed within the outer container and filled with the gas system, the compartment including an inner wall facing the cavity. The valve includes a hole formed in the inner wall and a tab a tab removably coupled to the inner wall such that the tab covers and seals the hole, and the tab is removable to open the hole and permit the gas system to diffuse from the compartment into the cavity.

The present disclosure provides, in another aspect, a system for storing a blood product including an inner container configured to contain the blood product, wherein the inner container is permeable to a gas system, an outer container including a sealable end and a cavity configured to receive the inner container through the sealable end prior to sealing the end, wherein the outer container is impermeable to the gas system, a compartment disposed within the outer container and filled with the gas system, the compartment including an inner wall facing the cavity and a hole formed in the inner wall, and a tab removably coupled to the inner wall such that the tab covers and seals the hole. The tab is removable to open the hole and permit the gas system to diffuse from the compartment into the cavity.

In some embodiments, the tab extends through the sealable end of the outer container.

In some embodiments, the compartment is integrally formed with the outer container.

The present disclosure provides, in another aspect, a method of storing a blood product, including inserting an inner container containing the blood product into a cavity of an outer container through an open end of the outer container, the inner container being permeable to a gas system and the outer container being impermeable to the gas system, opening a hole within the cavity, introducing the gas system into the cavity through the hole, and sealing the open end of the outer container to hermetically seal the inner container within the cavity of the outer container.

In some embodiments, the hole is located on a compartment within the cavity, the compartment containing the gas system, and opening the hole includes pulling on a tab extending through the open end of the outer container.

In some embodiments, sealing the open end includes partially heat sealing the open end prior to opening the hole, and fully heat sealing the open end after opening the hole.

Other features and aspects of the disclosure will become apparent by consideration of the following detailed description and accompanying drawings.

### BRIEF DESCRIPTION OF THE DRAWINGS

FIG. 1 is a top view of a storage system according to an embodiment of the present disclosure.

FIG. 2 is a schematic cross-sectional view of the storage system of FIG. 1.

FIG. 3 is an internal view illustrating a valve of the storage system of FIG. 1.

FIG. 4 is a schematic cross-sectional view illustrating an edge seal of the storage system of FIG. 1.

FIG. 5 is a schematic illustration of a storage system according to another embodiment of the present disclosure.

FIG. 5A is an enlarged detail view illustrating a valve of the storage system of FIG. 5.

FIG. 6 is a perspective view of a storage system according to an embodiment of the present disclosure.

FIG. 7 is a schematic cross-sectional view of the storage system of FIG. 6.

FIG. 8 is a perspective view illustrating heat sealing of an outer container of the storage system of FIG. 6.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways.

### DETAILED DESCRIPTION

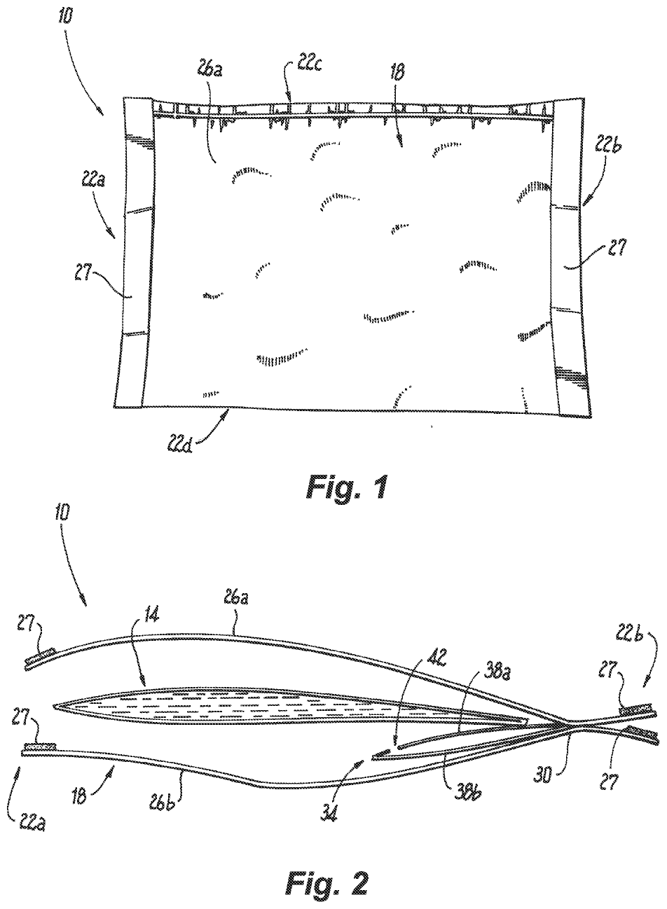

FIGS. 1-2 illustrate a storage system 10 according to an embodiment of the present disclosure. The storage system 10 includes a first or inner container 14 (FIG. 2) and a second or outer container 18 configured to receive the inner container 14 therein. In the illustrated embodiment, the outer container 18 is a flexible bag that includes a cavity sized and shaped to be able to fully contain the inner container 14 within the cavity of the outer container 18. The illustrated inner container 14 is configured as a flexible bag used to store a biological material, and more specifically, a blood product or cellular culture such as a platelet concentrate for later use in an organism.

The inner and outer containers 14, 18 of the illustrated storage system 10 are made of different materials or combinations of materials. More particularly, the inner container 14 is made of a flexible material that is permeable to a gas system, which in some embodiments may include xenon, a mixture of xenon and oxygen, or other gases suitable for enhancing preservation of the biological material contained within the inner container 14.

In some embodiments, the xenon content of the gas system is at least 5 vol. %. In some embodiments, the xenon content of the gas system is up to 99.99999 vol. %. In some embodiments, the xenon content of the gas system is at least 5 vol. % and up to about 99.99999 vol. % (e.g., 5 vol. %, 5.00001 vol. %, 5.00002 vol. % . . . 99.99998 vol. %, 99.99999 vol. %) and any value or range there between. In some embodiments, the xenon content of the gas system is from about 50-99.999 vol. %. In some embodiments, the xenon content of the gas system is from about 55-99 vol. %. In some embodiments, the xenon content of the gas system is from about 60-98 vol. %. In some embodiments, the xenon content of the gas system is from about 70-97 vol. %. In some embodiments, the xenon content of the gas system is from about 79-95 vol. %. In some embodiments, the oxygen content of the gas system is about 0-50 vol. % (e.g., 0 vol. %, 0.0001 vol. %, 0.0002 vol. % . . . 49.9998 vol. %, 49.9999 vol. %, 50 vol. %) and any value or range there between. In some embodiments, the oxygen content of the gas system is about 0.1-45 vol. %. In some embodiments, the oxygen content of the gas system is about 2-40 vol. %. In some embodiments, the oxygen content of the gas system is about 3-30 vol. %. In some embodiments, the oxygen content of the gas system is about 5-21 vol. %. In some embodiments, the gas system includes 0-5% by volume (e.g., 0%, 0.0001%, 0.0002% . . . 4.9998%, 4.9999%, 5%) and any value or range therebetween of a gas that is other than xenon or oxygen (e.g., carbon dioxide, noble gas, nitrogen). In some embodiments, the gas system of xenon, CO2 and optionally containing nitrogen. In some embodiments, the gas system includes at least 9 vol. % xenon (e.g., 9-99 vol. %), at least 1 vol. % CO2 (e.g., 1-10 vol. %) and optionally N2 (e.g., 0-90 vol. %). In another embodiment, the gas system includes at least 95 vol. % xenon (e.g., 9-99 vol. %), at least 1 vol. % nitrogen and/or CO2. In some embodiments, xenon volume percent is greater than the volume percent of CO2, and the nitrogen volume content, when included, can be greater than or less than the volume content CO2.

The outer container 18 is made of a material and/or includes a film or coating that is impermeable to the gas system. For example, in some embodiments, the outer container 18 is not permeable to xenon and any of the primary components of air (e.g., oxygen, nitrogen, carbon dioxide, water vapor, etc.). In some embodiments, the outer container 18 is made of one or more layers of thin film material, such as one or more layers of metal foil (e.g., aluminum foil or the like).

Referring to FIG. 1, the outer container 18 includes a first end 22a, a second end 22b opposite the first end 22a, and first and second sides 22c, 22d extending between the ends 22a, 22b. The outer container 18 includes first and second sheets 26a, 26b joined and sealed together at the sides 22c, 22d and at the second end 22b (e.g., by heat sealing, an adhesive, or the like) (FIG. 2). Generally, the outer container 18 is open at the first end 22a so that the inner container 14 can be inserted into the cavity of the outer container 18. It should be understood, however, that any of the ends 22a, 22b or sides 22c, 22d of the outer container 18 may define the opening into the outer container 18, with the other ends 22a, 22b and sides 22c, 22d being sealed. Furthermore, in some embodiments, the first and second sheets 26a, 26b may be portions of a single contiguous sheet of material folded over at one of the ends 22a, 22b or sides 22c, 22d.

Once the inner container 14 is inserted into the outer container 18, the open end or side of the outer container 18 (i.e., the first end 22a in the illustrated embodiment) can be sealed. For example, in the illustrated embodiment, the first end 22a is provided with an adhesive strip 27 located on each of the first and second sheets 26a, 26b (FIG. 2). The adhesive strips 27 are pressure sensitive, such that pressing the adhesive strips 27 of the sheets 26a, 26b together seals the first end 22a of the outer container 18. In some embodiments, the first end 22a may be folded over—to define a fold 28—before pressing the adhesive strips 27 together, which may improve the strength of the seal (FIG. 4). In yet other embodiments, the first end 22a may additionally or alternatively be sealed by welding the sheets 26a, 26b together, or via any other suitable method. In some such embodiments, the adhesive strips 27 may initially hold the first end 22a closed to facilitate subsequent welding. Once the first end 22a of the outer container 18 is closed and sealed, the cavity of the outer container 18 is hermetically sealed.

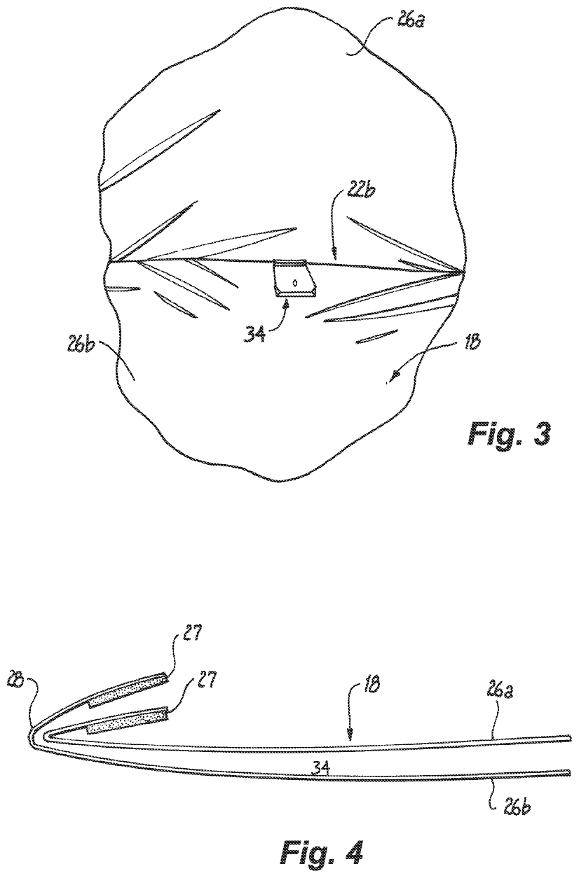

With reference to FIGS. 2-3, the outer container 18 includes an inlet 30 and a filling valve 34 for controlling gas flow through the inlet 30. In the illustrated embodiment, the inlet 30 and filling valve 34 are located at the second end 22b of the outer container 18. In some embodiments, the inlet 30 may extend through a seam in the second end 22b of the outer container 18. The illustrated filling valve 34 extends from the inlet 30 and into the cavity of the outer container 18. The filling valve 34 includes first and second sheets of material 38a, 38b, bonded together at their perimeters to form sleeve or tube having an open end facing away from the cavity to define the inlet 30 and a sealed end opposite the inlet 30. The sheets of material 38a, 38b may be made of the same material as the sheets 26a, 26b forming the outer container 18, which simplifies construction of the outer container 18 and filling valve 34. In other embodiments, the filling valve 34 may be formed from a single sheet of material rolled into a sleeve or tube shape and sealed.

The filling valve 34 includes a perforation 42 located within the chamber of the outer container 18. As described in more detail below, a gas system introduced through the inlet 30 may flow into the outer container 18 through the perforation 42.

In use, the cavity of the inner container 14 is filled with the biological material to be preserved. The biological material is sealed in the inner container 14, the inner container 14 is inserted into the cavity of the outer container 18, through the open first end 22a. After the inner container 14 is inserted into the cavity of the outer container 18, the cavity of the outer container 18 is hermetically sealed by sealing the first end 22a (e.g., by the adhesive strips 27, by heat sealing, or any other suitable means for forming a hermetic seal). The cavity of the outer container 18 is configured such that the inner container 14 does not need to be opened or otherwise have the integrity of the inner container 14 compromised when the inner container 14 is placed in the outer container 18.

After the inner container 14 is inserted into the cavity of the outer container 18 and after the hermetic sealing of the cavity of the outer container 18 while the cavity fully contains the inner container 14, a gas system is added to the cavity of the outer container 18, via the inlet 30. For example, in some embodiments, the inlet 30 can be connected to a gas filling tube which is in turn connected to a source of the gas system.

The valve 34 allows the gas system to freely flow into the cavity of the outer container 18. Since the gas system at the source is at a higher pressure than the cavity, the gas pressure of the gas system inflates the valve 34 and allows the gas to travel through the valve 34 (i.e. between the sheets 38a, 38b), through the perforation 42, and ultimately into the cavity of the outer container 18. In some embodiments, the cavity is filled with the gas system to a pressure about 0.5-5 bars above atmospheric pressure (e.g., 1 atm.).

Once the cavity of the outer container 18 is pressurized to the desired pressure, the source of the gas system is disconnected from the inlet 30. The gas backpressure inside the cavity of the outer container 18 causes the valve 34 to collapse, thereby preventing the gas system from escaping the cavity of the outer container 18. The second end 22b of the outer container 18 may then optionally be sealed (such as by heat sealing or another suitable method) to permanently seal the valve 34.

In some embodiments, after filling the cavity of the outer container 18 with the gas system, the storage system 10 may be placed into a pressure chamber and exposed to an elevated pressure and/or refrigerated storage environment, such as according to the methods described in U.S. Publication No. 2018/0249703 noted above. By increasing the pressure around the outer container 18, the pressure of the gas system contained within the outer container 18 may be increased due to the flexible construction of the outer container 18, increasing the amount of the gas system that permeates into the biological material contained within the inner container 14.

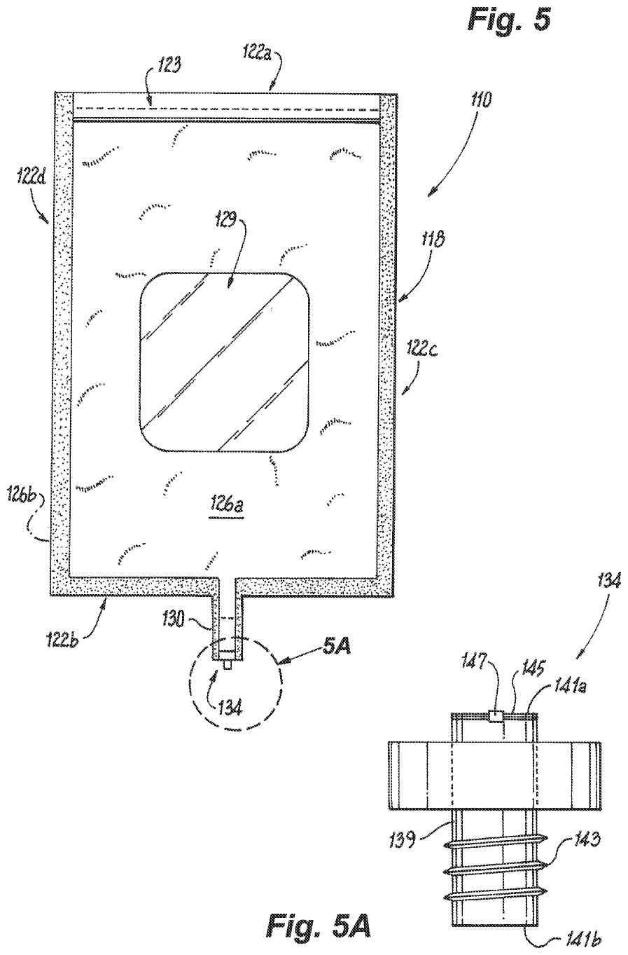

FIG. 5 illustrates a storage system 110 according to another embodiment of the present disclosure. The storage system 110 is similar to the storage system 10 described above with reference to FIGS. 1-4, and features of the storage system 110 corresponding to features of the storage system 10 are given like reference numerals plus ‘100.’ The following description focuses primarily on differences between the storage system 110 and the storage system 10, and it should be understood that features and alternatives of the storage system 10 described above may be incorporated into the storage system 110 and vice versa.

The storage system 110 includes a second or outer container 118 configured to receive an inner container (such as the inner container 14; FIG. 2) therein. Referring to FIG. 5, the outer container 118 includes a first end 122a, a second end 122b opposite the first end 122a, and first and second sides 122c, 122d extending between the ends 122a, 122b. The outer container 118 is formed from first and second sheets 126a, 126b joined together at the sides 122c, 122d and at the second end 122b (e.g., by heat sealing, an adhesive, or the like). In some embodiments, the first and second sheets 126a, 126b may be portions of a single contiguous sheet of material folded over.

In the illustrated embodiment, the outer container 118 includes an interlocking closure 123 (e.g., a zip-locking closure) extending along a width of the first end 122a. The closure 123 allows the first end 122a of the outer container 118 to be opened to permit the inner container to be inserted inside the outer container 118, then closed to seal the inner container within the cavity of the outer container 118.

With continued reference to FIG. 5, in the illustrated embodiment, the first sheet 126a of the outer container 118 includes a window 129. The window 129 is transparent, such that a user can visually determine whether the inner container is present within the outer container 118. The illustrated window 129 is centered on the outer container 118, allowing a user to observe and read identifying information, such as labels, barcodes, or the like, placed on the inner container. In some embodiments, the inner container or at least a portion thereof may also be transparent, so that the user may observe the contents of the inner container through the window 129 in the outer container 118 (e.g., to assess the condition of the contents of the inner container). Like the remainder of the outer container 118, the window 129 is impermeable to the gas system that may be inserted into the outer container 118, as described below.

The second end 122b of the outer container 118 may include an inlet 130 and a filling valve 134 for controlling gas flow through the inlet 130. The filling valve 134 may be positioned within the inlet 130 and/or affixed to the inlet 130 in any suitable manner. In the illustrated embodiment, the inlet 130 extends from the second end 122b of the outer container 118 to define an elongated air channel. In other embodiments, the inlet 130 may extend through a seam in the second end 122b of the outer container 118 without extending beyond the second end 122b.

With reference to FIG. 5A, the illustrated valve 134 includes a tube 139 with a first end 141a facing the inlet 130 and a second end 141b opposite the first end 141a. The tube 139 has external threads 143 extending generally from the second end 141b, which are configured for connection to a source of the gas system (e.g., to a regulator on a pressurized gas cylinder containing the gas system). The first end 141a of the tube 139, which is disposed within the inlet 130 in the illustrated embodiment, is covered with a flexible membrane 145. The membrane 145 includes a small orifice or pinhole 147. As described in greater detail below, the gas system may be introduced through the valve 134 and flow into the outer container 118 through the orifice 147.

In use, the cavity of the inner container (e.g., inner container 14; FIG. 2) is filled with the biological material prior to being sealed. After the biological material is sealed in the inner container 14, the inner container is inserted into the cavity of the outer container 118, through the open first end 122a. After the inner container is inserted into the cavity of the outer container 118, the cavity of the outer container 118 is sealed by pressing on the interlocking closure 134. The closure 134 alone may form a hermetic seal, but the closure 134 also advantageously aligns the first and second sheets 126a, 126b at the first end 122a to facilitate a subsequent heat sealing operation to provide a permanent hermetic seal.

After the inner container is hermetically sealed within the cavity of the outer container 118, the gas system is added to the cavity of the outer container 118, via the valve 134 and the inlet 130. When the pressurized gas system is introduced into the valve 134, the membrane 145 deforms outwardly, which enlarges the orifice 147 and permits the gas system to flow through the valve 134 and into the outer container 118 via the inlet 130. In some embodiments, the cavity is filled with the gas system to a pressure about 0.5-5 bars above atmospheric pressure (e.g., 1 atm.).

Once the cavity of the outer container 118 is pressurized to the desired pressure, the source of the gas system is disconnected from the valve 134. The gas backpressure inside the cavity of the outer container 118 causes the membrane 145 to contract, which in turn seals the orifice 147. The inlet 130 of the outer container 118 may then optionally be sealed with an additional sealing step (such as by heat sealing or another suitable method) to form a permanent seal.

In some embodiments, after filling the cavity of the outer container 118 with the gas system, the storage system 110 may be placed into a pressure chamber and exposed to an elevated pressure and/or refrigerated storage environment, such as according to the methods described in U.S. Publication No. 2018/0249703 noted above. By increasing the pressure around the outer container 118, the pressure of the gas system contained within the outer container 118 may be increased due to the flexible construction of the outer container 118, increasing the amount of the gas system that permeates into the biological material contained within the inner container.

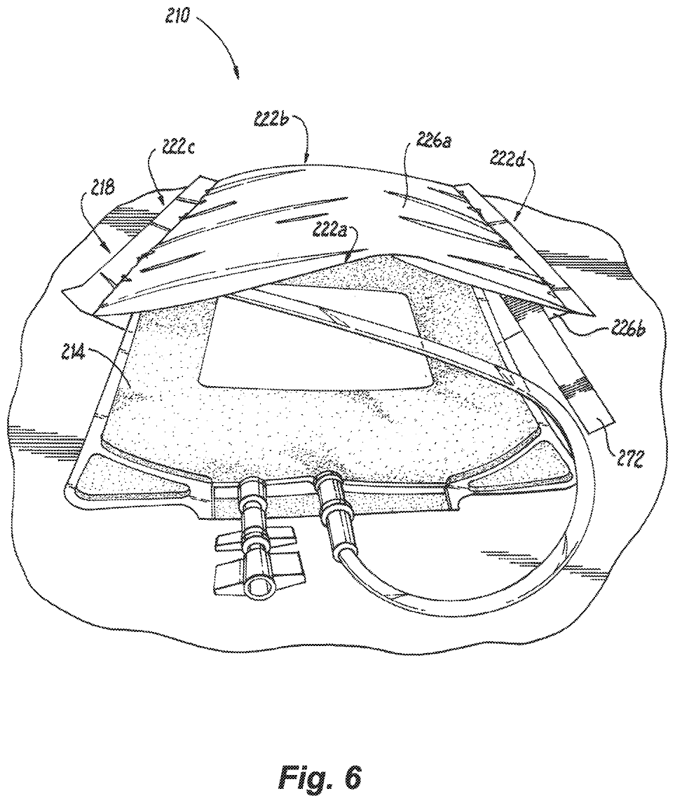

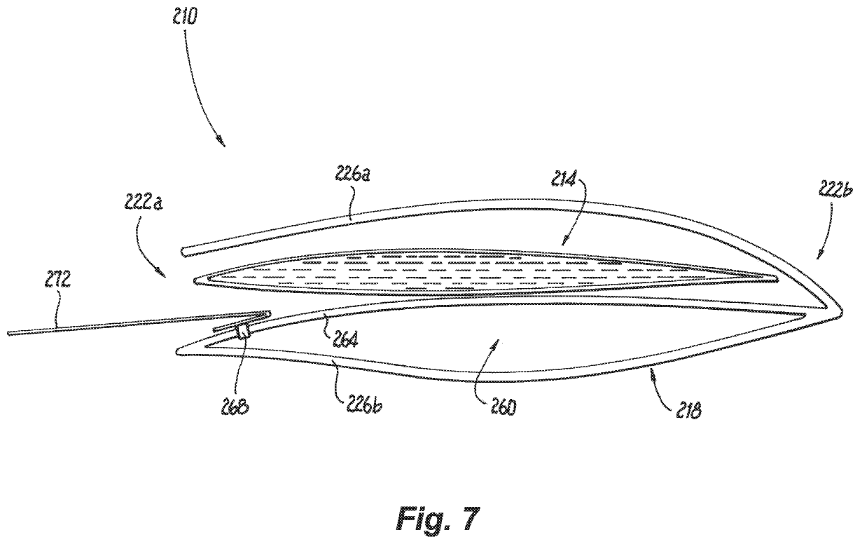

FIGS. 6 and 7 illustrate a storage system 210 according to another embodiment of the present disclosure. The storage system 210 is similar to the storage system 10 described above, and features and elements of the storage system 210 corresponding to features and elements of the storage system 10 are given like reference numerals plus ‘200.’ The following description focuses primarily on differences between the storage system 210 and the storage system 10, and it should be understood that features and alternatives of the storage system 10 and the storage system 110 described above may be incorporated into the storage system 210 and vice versa.

The storage system 210 includes a first or inner container 214 and a second or outer container 218 configured to receive the inner container 214 therein. In the illustrated embodiment, the outer container 218 is a flexible bag that includes a cavity that is sized and shaped to be able to fully contain the inner container 214 within the cavity of the outer container 218. The illustrated inner container 214 is a flexible bag used to store blood products and/or cellular cultures, such as platelet concentrates.

Referring to FIG. 6, the outer container 218 includes a first end 222a, a second end 222b opposite the first end 222a, and first and second sides 222c, 222d extending between the ends 222a, 222b. The outer container 218 includes first and second sheets 226a, 226b joined together at the sides 222c, 222d and at the second end 222b (e.g., by heat sealing or an adhesive). Generally, the outer container 218 is open at the first end 222a so that the inner container 214 can be inserted into the cavity of the outer container 218. Thereafter, the first end 222a of the outer container 218 can be sealed. It should be understood, however, that any of the ends 222a, 222b or sides 222c, 222d of the outer container 218 may define the opening into the outer container 218, with the other ends 222a, 222b and sides 222c, 222d being sealed. Furthermore, in some embodiments, the first and second sheets 226a, 226b may be portions of a single contiguous sheet of material folded over at one of the ends 222a, 222b or sides 222c, 222d.

For example, the first end 222a may be provided with an adhesive strip located on each of the first and second sheets 226a, 226b. Pressing the adhesive strips of the sheets 226a, 226b together seals the first end 222a of the outer container 218. In some embodiments, the first end 222a may be folded over before pressing the adhesive strips together, which may improve the strength of the seal. In yet other embodiments, the first end 222a may be sealed by welding the sheets 226a, 226b together, or via any other suitable method. In some such embodiments, the adhesive strips may initially hold the first end 222a closed to facilitate subsequent welding. Once the first end 222a of the outer container 218 is closed and sealed, the cavity of the outer container 218 is hermetically sealed.

Referring to FIG. 7, the illustrated outer container 218 includes a hermetically sealed insert or compartment 260 containing a pressurized volume of a gas system, such as the gas system described above. The compartment 260 may be integrally formed as a part outer container 218 (e.g., by folding over and sealing a layer of the container 218). A hole 268 is formed in an interior wall 264 of the compartment 260. The hole 268 is covered by a removable tab 272 with a length that extends through the first end 222a of the outer container 218. The tab 272 hermetically seals the hole 268 in order to maintain the gas system within the compartment 260. As such, the compartment 260 can be pre-filled with the gas system during manufacturing of the outer container 218. In some embodiments, the gas system may be introduced into the compartment 260 through the hole 268 prior to applying the tab 272 over the hole 268 to seal the hole 268.

In use, the cavity of the inner container 214 is filled with the biological material to be preserved. After the biological material is sealed in the inner container 214, the inner container 214 is inserted into the cavity of the outer container 218, through the open first end 222a. The cavity of the outer container 218 is configured such that the inner container 214 does not need to be opened or otherwise have the integrity of the inner container 214 compromised when the inner container 214 is placed in the outer container 218.

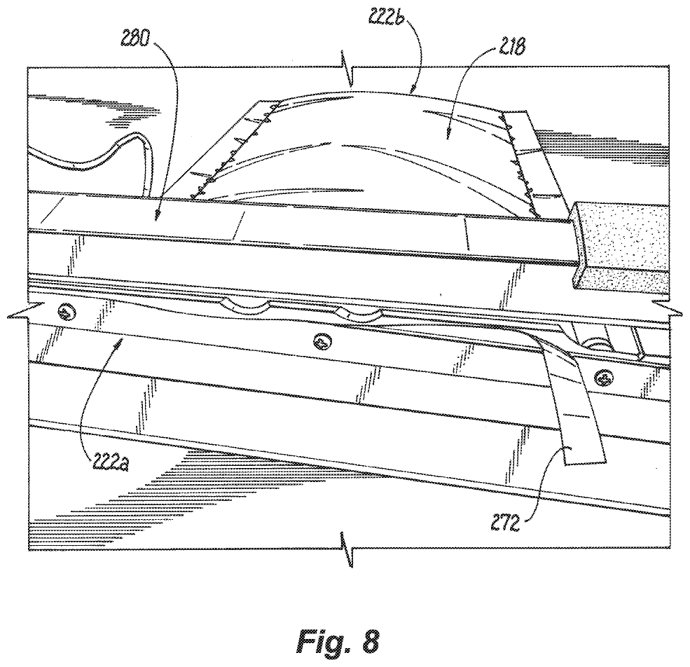

After the inner container 214 is inserted into the cavity of the outer container 218, the cavity of the outer container 218 is hermetically sealed. For example, as illustrated in FIG. 8, the first end 222a of the outer container 218 may be placed in a heat sealing machine 280. Either during or just prior to sealing, an operator removes the tab 272 by pulling on the exposed end of the tab 272 that extends through the first end 222a of the outer container 218. The tab 272 detaches from the interior wall 264 of the compartment 260, which opens the hole 268 and allows the gas system to flow out of the compartment 260 and into the internal cavity of the outer container 218. In this way, the tab 272 and hole 268 define a valve for controlling flow of the gas system into the cavity of the outer container 218. The first end 222a is then fully sealed to hermetically seal the internal cavity. In other embodiments, the first end 222a of the outer container 218 may be sealed using other suitable means, such as adhesives.

The gas system from the compartment 260 diffuses into the cavity of the outer container 218 and is able to permeate through the inner container 214 and into the biological material contained within the inner container 214. Because the gas system is able to be introduced into the compartment 260 and sealed with the tab 272 during manufacturing of the outer container 218, there is no need for an on-site filling container for introducing the gas system into the outer container 218. This makes the storage system 210 versatile to use in a variety of settings, where bulk supplies of the gas system may be unavailable.

In some embodiments, after removing the tab 272 and sealing the first end 222a of the outer container 218, the storage system 210 may be placed into a pressure chamber and exposed to an elevated pressure and/or refrigerated storage environment, such as according to the methods described in U.S. Publication No. 2018/0249703 noted above. By increasing the pressure around the outer container 218, the pressure of the gas system contained within the outer container 218 may be increased due to the flexible construction of the outer container 218, increasing the amount of the gas system that permeates into the biological material contained within the inner container 214.

Various features of the invention are set forth in the following claims.

What is claimed is:

1. A system for storing a blood product comprising:
an inner container configured to contain the blood product, wherein the inner container is permeable to a gas system; and
an outer container including
a first end,
a second end opposite the first end,
a cavity defined between the first and second ends,
an inlet in fluid communication with the cavity, and
a valve operable to control a flow of the gas system through the inlet,
wherein the inner container is insertable into the cavity through the first end,
wherein the first end is sealable to hermetically seal the inner container within the cavity, and
wherein the outer container is made of a material that is impermeable to the gas system.

2. The system of claim 1, wherein the valve includes a sleeve extending into the cavity and having a perforation, and wherein the gas system is configured to flow into the cavity through the sleeve and the perforation when a gas system source is connected to the inlet.

3. The system of claim 2, wherein the sleeve is configured to collapse when a pressure within the cavity is greater than a pressure at the inlet.

4. The system of claim 1, wherein the outer container includes a first sheet of material and a second sheet of material sealed together at the second end, and wherein the inlet extends through the second end.

5. The system of claim 1, wherein the valve includes a flexible membrane and a orifice formed in the flexible membrane.

6. The system of claim 5, wherein the membrane is configured to expand to open the orifice when a gas system source is connected to the inlet.

7. The system of claim 1, wherein the outer container includes a transparent window.

8. The system of claim 7, wherein the inner bag is visible through the transparent window when the inner bag is hermetically sealed within the cavity.

9. The system of claim 1, wherein the valve includes external threads.

10. The system of claim 1, wherein the first end of the outer container includes an interlocking closure.

11. The system of claim 1, wherein the gas system includes xenon.

12. The system of claim 1, wherein the outer container comprises metal foil.

13. The system of claim 1, wherein the valve is sealed inside the cavity when the first end is sealed.

14. The system of claim 1, further comprising a compartment disposed within the outer container and filled with the gas system, the compartment including an inner wall facing the cavity,
wherein the valve includes a hole formed in the inner wall and a tab a tab removably coupled to the inner wall such that the tab covers and seals the hole, and
wherein the tab is removable to open the hole and permit the gas system to diffuse from the compartment into the cavity.

15. A system for storing a blood product comprising:
an inner container configured to contain the blood product, wherein the inner container is permeable to a gas system;
an outer container including a sealable end and a cavity configured to receive the inner container through the sealable end prior to sealing the end, wherein the outer container is impermeable to the gas system;
a compartment disposed within the outer container and filled with the gas system, the compartment including an inner wall facing the cavity and a hole formed in the inner wall; and
a tab removably coupled to the inner wall such that the tab covers and seals the hole,
wherein the tab is removable to open the hole and permit the gas system to diffuse from the compartment into the cavity.

16. The system of claim 15, wherein the tab extends through the sealable end of the outer container.

17. The system of claim 15, wherein the compartment is integrally formed with the outer container.

18. A method of storing a blood product, comprising:
inserting an inner container containing the blood product into a cavity of an outer container through an open end of the outer container, the inner container being permeable to a gas system and the outer container being impermeable to the gas system;
opening a hole within the cavity;
introducing the gas system into the cavity through the hole; and
sealing the open end of the outer container to hermetically seal the inner container within the cavity of the outer container.

19. The method of claim 18, wherein the hole is located on a compartment within the cavity, the compartment containing the gas system, and wherein opening the hole includes pulling on a tab extending through the open end of the outer container.

20. The method of claim 19, wherein sealing the open end includes partially heat sealing the open end prior to opening the hole, and fully heat sealing the open end after opening the hole.
